(19) United States
(12) Patent Application Publication
(10) Pub. No.: US 2026/0355046 A1
(43) Pub. Date: Nov. 12, 2026

(54) EYE MASSAGER

(71) Applicant: Reestar International Limited, Hong Kong (CN)

(72) Inventors: Gaoming Liang, Hong Kong (CN); Kiu Chan, Hong Kong (CN); Xili Wu, Hong Kong (CN); Wesley King Juen Tong, Hong Kong (CN); Wai Lik Tsoi, Hong Kong (CN)

(21) Appl. No.: 19/635,782

(22) Filed: Apr. 1, 2026

Related U.S. Application Data
(60) Provisional application No. 63/803,392, filed on May 9, 2025.

(30) Foreign Application Priority Data
Dec. 19, 2025 (CN) 202522705270.9

(51) Int. Cl. A61H 9/00; A61F 7/00; A61F 7/02

(52) U.S. Cl. CPC A61H 9/0078; A61F 7/007; A61F 7/02; A61F 2007/0004; A61F 2007/0093; A61F 2007/0095; A61H 2201/0207; A61H 2201/0214; A61H 2201/0285; A61H 2201/1238; A61H 2201/165; A61H 2201/5007; A61H 2201/5082; A61H 2205/024

(57) ABSTRACT

An eye massager includes a body assembly. The body assembly includes a body housing, a cold and hot compress assembly, and a heat dissipation assembly. The body housing defines at least one first air vent and at least one second air vent and includes an inner face, a first housing, and a second housing. The at least one first air vent functions as an air inlet, and the at least one second air vent functions as an air outlet. The cold and hot compress assembly includes a thermoelectric member and a heat conduction member. The thermoelectric member includes a first working portion. The heat conduction member is configured to at least partially exchange heat with the first working portion, and apply cold or hot compress to the user. The heat dissipation assembly is configured to dissipate heat from the cold and hot compress assembly.

### CROSS REFERENCE TO RELATED APPLICATIONS

This application claims priority of the US provisional patent application No. 63/803,392, filed on May 9, 2025, and claims priority of the Chinese patent application No. 202522705270.9, filed on Dec. 19, 2025. The disclosure of which is hereby incorporated by reference in their entireties.

### FIELD OF THE DISCLOSURE

The present disclosure relates to the field of massage devices, and in particular, to an eye massager.

### BACKGROUND

Modern people, due to prolonged use of electronic products such as computers, mobile phones, game consoles, or due to poor sleep quality, are prone to eye fatigue, dark circles, eye wrinkles, and other symptoms.

Cold compress can reduce local tissue temperature, causing vasoconstriction, slowed nerve conduction, and slowed metabolism, thereby producing effects such as pain relief, swelling reduction, and inflammation suppression.

Hot compress can increase local tissue temperature, causing vasodilation, accelerating metabolism and circulation, and promoting repair and relaxation.

Cold compress bags cooled by freezer have significant limitations, lack freedom in timing, and may be inconvenient to carry for outdoor use.

### SUMMARY

The following presents a simplified summary of the disclosure in order to provide a basic understanding of some aspects of the disclosure. This summary is not an extensive overview of the disclosure. It is not intended to identify critical elements or to delineate the scope of the disclosure. Its sole purpose is to present some concepts of the disclosure in a simplified form as a prelude to the more detailed description that is presented elsewhere.

In some embodiments, an eye massager is provided. The eye massager includes at least one body assembly. The at least one body assembly includes a body housing, a cold and hot compress assembly, and a heat dissipation assembly. The body housing defines at least one first air vent and at least one second air vent and includes an inner face, a first housing, and a second housing. The first housing is mounted to an outer side of the second housing, and at least one of the first air vent and the second air vent is defined by a first gap formed between the first housing and the second housing. The at least one first air vent functions as an air inlet, and the at least one second air vent functions as an air outlet. The cold and hot compress assembly includes a thermoelectric member and a heat conduction member. The thermoelectric member includes a first working portion. Changing a current direction in the thermoelectric member enables the first working portion to cool or heat. The heat conduction member is configured to at least partially exchange heat with the first working portion, and apply cold or hot compress to the user. The heat dissipation assembly is at least partially arranged in the body housing, and configured to dissipate heat from the cold and hot compress assembly.

### BRIEF DESCRIPTION OF THE DRAWINGS

Illustrative embodiments of the present disclosure are described in detail below with reference to the attached drawing figures.

FIG. 1 is a structural view of an eye massager according to an embodiment of the present disclosure.

FIG. 2 is a structural view of the eye massager from another perspective according to an embodiment of the present disclosure.

FIG. 3 is an exploded view of a portion of the eye massager according to an embodiment of the present disclosure.

FIG. 4 is an exploded view of another portion of the eye massager according to an embodiment of the present disclosure.

FIG. 5 is a schematic view of an internal structure of a body housing according to an embodiment of the present disclosure.

FIG. 6 is a structural view of the body housing, a cold and hot compress assembly, a heat dissipation assembly, and an air bag according to an embodiment of the present disclosure.

FIG. 7A is a front view of a body assembly according to an embodiment of the present disclosure.

FIG. 7B is a three-dimensional cross-sectional view taken along a line A-A in FIG. 7A.

FIG. 7C is a three-dimensional cross-sectional view taken along a line B-B in FIG. 7A.

FIG. 7D is another three-dimensional cross-sectional view taken along the line A-A in FIG. 7A.

FIG. 7E is another three-dimensional cross-sectional view taken along the line B-B in FIG. 7A.

FIG. 8A is an exploded view of the cold and hot compress assembly according to an embodiment of the present disclosure.

FIG. 8B is an exploded view of the cold and hot compress assembly from another perspective according to an embodiment of the present disclosure.

FIG. 9A is an exploded view of a first housing and the body housing according to an embodiment of the present disclosure.

FIG. 9B is an exploded view of the first housing and the body housing from another perspective in a same embodiment as FIG. 9A.

FIG. 9C is a front view of the first housing and the body housing in the same embodiment as FIG. 9A.

FIG. 9D is a three-dimensional cross-sectional view taken along line C-C in FIG. 9C.

FIG. 10A is an exploded view of the first housing and the body housing according to another embodiment of the present disclosure.

FIG. 10B is an exploded view of the first housing and the body housing from another perspective in a same embodiment as FIG. 10A.

FIG. 10C is a front view of the first housing and the body housing in the same embodiment as FIG. 10A.

FIG. 10D is a three-dimensional cross-sectional view taken along line D-D in FIG. 10C.

FIG. 11A is an exploded view of the first housing and the body housing according to another embodiment of the present disclosure.

FIG. 11B is a front view of the first housing and the body housing in a same embodiment as FIG. 11A.

FIG. 11C is a three-dimensional cross-sectional view taken along line E-E in FIG. 11B.

FIG. 12 is a functional block diagram of the eye massager according to an embodiment of the present disclosure.

### REFERENCE NUMERALS IN THE DRAWINGS

1-body assembly; 11-body housing; 111-first housing; 1111-outer face; 112-second housing; 1121-bearing face; 11211-opening; 112111-first opening; 112112-second opening; 1122-flow guide member; 11221-flow guide face; 113-third housing; 1131-peripheral side wall; 1132-inner face; 1133-button; 1134-outer face; 114-first air vent; 115-second air vent; 116-first gap; 117-accommodation cavity; 118-second gap; 1181-second air passage; 1182-third air passage; 119-shielding portion; 1110-fastening member; 12-cold and hot compress assembly; 121-thermoelectric member; 1210-first working portion; 1211-first face; 1212-second face; 122-base; 123-heat conduction member; 1231-working face; 13-support member; 131-contact layer; 132-elastic member; 1321-first end; 1322-second end; 1323-telescopic sleeve; 13231-side wall; 13232-first air passage; 14-heat dissipation assembly; 141-fan; 142-heat-dissipating member; 2-massage assembly; 20-inflation/deflation assembly; 21-air pump; 22-air valve; 23-nozzle; 24-air bag; 4-battery; 5-speaker; 6-strap; 7-controller; 8-memory; 9-detection member; 10-voice assembly; 101-vibration motor.

### DETAILED DESCRIPTION

The following describes some non-limiting exemplary embodiments of the disclosure with reference to the accompanying drawings. The described embodiments are merely a part rather than all of the embodiments of the disclosure. All other embodiments obtained by a person of ordinary skill in the art based on the embodiments of the disclosure shall fall within the scope of the disclosure.

Unless otherwise defined, all technical and scientific terms used herein have the same meaning as commonly understood by those skilled in the art to which the present disclosure belongs. The terms used in the description of the present disclosure herein are intended for describing particular embodiments only and are not intended to limit the present disclosure. In the description, claims, and the above drawings of the present disclosure, the terms “including” and “having”, as well as their variants, are intended to convey a non-exclusive inclusion. The terms “first”, “second”, etc., as used herein, are intended to distinguish between different objects, rather than to describe a particular order.

Reference to “embodiments” herein implies that a particular feature, structure, or characteristic described in conjunction with an embodiment may be included in at least one embodiment of the present disclosure. The appearance of the phrase at various places in the specification does not necessarily refer to the same embodiment, nor is it a separate or an alternative embodiment that is mutually exclusive of other embodiments. One skilled in the art would explicitly and implicitly understand that the embodiments described herein can be combined with other embodiments.

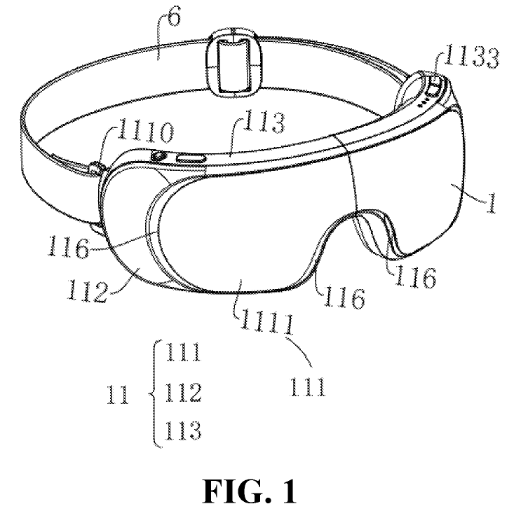

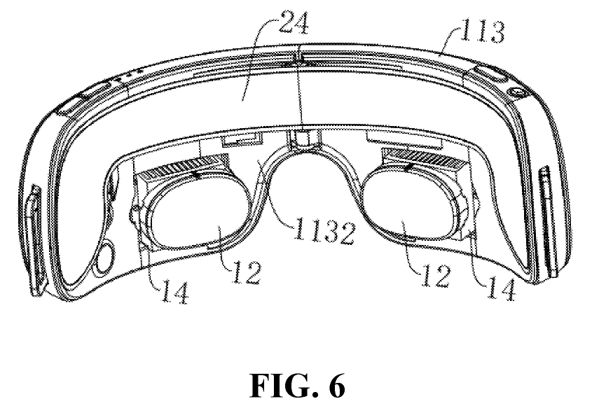

To solve the above technical problem, referring to FIGS. 1, 2, 3, 4, 6, 8A, and 8B, the present disclosure provides an eye massager, including two body assemblies 1. The two body assemblies 1 may be connected to each other by a hinge, capable of folding and unfolding relative to each other. Two cold and hot compress assemblies 12 may be provided, and each cold and hot compress assembly 12 may be arranged corresponding to a respective one of two periorbital areas of a human body. Two heat dissipation assemblies 14 may also be correspondingly provided. The body assembly 1 may include a body housing 11, a cold and hot compress assembly 12, and a heat dissipation assembly 14. The body housing 11 may include an inner face 1132 used to face the human body. The inner face 1132 may be curved, and a curvature of the inner face 1132 may match a facial contour from a nose to an ear. Structures of the two body assemblies 1 may be identical, and each body assembly 1 may include an inner face 1132. When the two body assemblies 1 are unfolded relative to each other, two inner faces 1132 may be joined each other to form an extended curved face, conforming to a curvature of a contour of an overall facial width of the human body. Curvatures of the two inner faces 1132 may be designed based on three-dimensional contour curves formed by cheekbones, brow bones, and nasal bridge of most users.

The two cold and hot compress assemblies 12 may be respectively arranged corresponding to the two eyes and periorbital areas of the human body. Each cold and hot compress assembly 12 may include a thermoelectric member 121 and a heat conduction member 123. The thermoelectric member 121 may include a first working portion 1210. The thermoelectric member 121 may be a thermoelectric cooler (TEC) which is a semiconductor, utilizing the Peltier effect, i.e., switching a direction of current through the thermoelectric member 121 to cause the first working portion 1210 to cool or heat. The heat conduction member 123 may be a metal sheet, particularly aluminum alloy material, and may also be a metal-like material or ceramic material. The heat conduction member 123 can at least partially exchange heat with the first working portion 1210, and the first working portion 1210 may transfer therapeutic heat or cold to the heat conduction member 123. A side of the heat conduction member 123 facing away from the first working portion 1210 can exchange heat with a respective periorbital area, specifically contacting an eye bag. The therapeutic heat or cold of the heat conduction member 123 may diffuse to eye area such as eyeball, to apply cold or hot compress to the eye and periorbital area. The heat dissipation assembly 14 may dissipate excess heat generated by the first working portion 1210, maintaining stable operation of the thermoelectric member 121 and quickly achieving switching between cold and hot compress.

Referring to FIGS. 1, 2, 3, 4, 9B, and 10B, the body assembly 1 further may include a support member 13. The support member 13 may be mounted to the body housing 11. The cold and hot compress assemblies 12 may be directly or indirectly mounted to the support member 13. The support member 13 may be deformable, thereby driving the cold and hot compress assemblies 12 to move. There may be significant differences in eye socket depth, nose bridge height, and eye socket shape among different individuals, and after the user wears the eye massager, the user’s eyes and periorbital areas may exert pressure on the cold and hot compress assemblies 12. At this time, the support member 13 may deform under the pressure, causing the cold and hot compress assemblies 12 to undergo slight movement and deflection along with the deformation of the support member 13, to dynamically adjust positions and angles of the cold and hot compress assemblies 12 relative to the user’s eyes and periorbital areas, ultimately achieving adaptive fitting of the cold and hot compress assemblies 12, specifically the heat conduction members 123 in the cold and hot compress assemblies 12, with the user’s eye and periorbital areas.

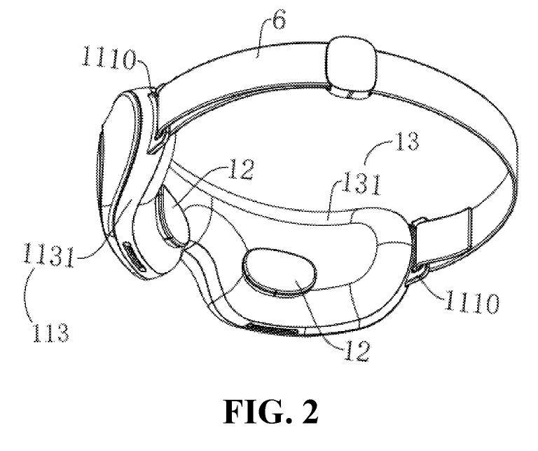

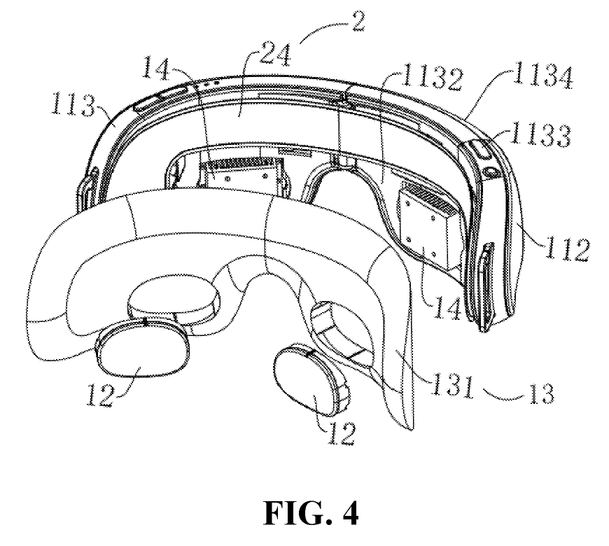

Referring to FIGS. 2 and 4, in some embodiments, the support member 13 may be a flexible contact layer 131. The body housing 11 may include an inner face 1132 facing the human body. The contact layer 131 may be attached to the inner face 1132 of the body housing 11. The cold and hot compress assemblies 12 may be at least partially embedded in the contact layer 131. After the cold and hot compress assemblies 12 receive the pressure exerted by the eyes and periorbital areas, the contact layer 131 may deform. Specifically, the contact layer 131 may undergo an elastic compression, causing a local reduction in a thickness of the contact layer 131, thereby allowing the cold and hot compress assembly 12 to displace in a direction of the elastic compression of the contact layer 131. Due to curved characteristics of contours of eyes and periorbital areas and the properties of the material of the contact layer 131, the contact layer 131 may undergo lateral stretching or contraction at portions connected with the cold and hot compress assemblies 12, allowing the cold and hot compress assemblies 12 to undergo slight displacement and deflection. In this way, the cold and hot compress assemblies 12 can dynamically adjust positions and angles of the cold and hot compress assemblies 12 contact with the eyes and periorbital areas, to match concave and convex contour of the eyes and periorbital areas of different users.

The contact layer 131 may include an outer fabric layer that may contact human skin, and a filler filled in the outer fabric layer. The outer fabric layer may be protein leather to provide a skin-friendly touch. The filler may be sponges of different thicknesses, such as slow-rebound memory foam or high-elasticity foam. Thinner sponges may be used at protruding parts of the user’s eyes and periorbital areas, and thicker sponges may be used at recessed parts of the user’s eyes and periorbital areas. Through thickness differences of sponges in different areas, the contact layer 131 and the cold and hot compress assemblies 12 can better fit the user’s eyes and periorbital areas.

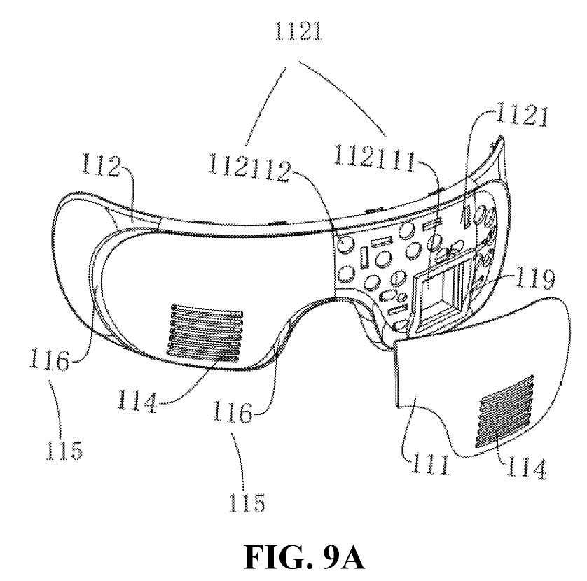

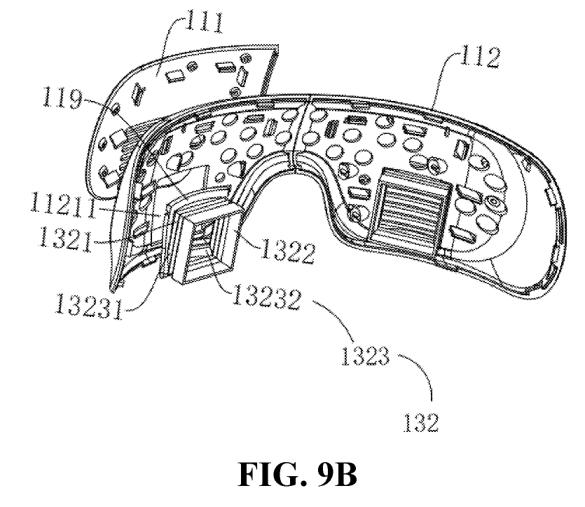

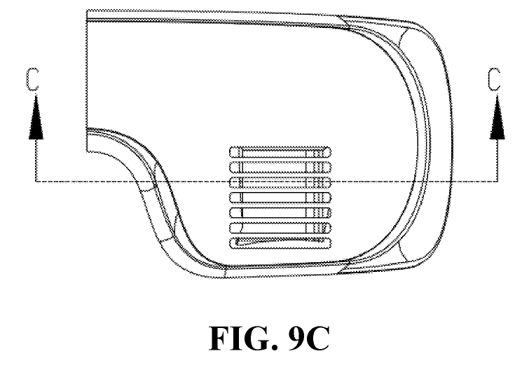

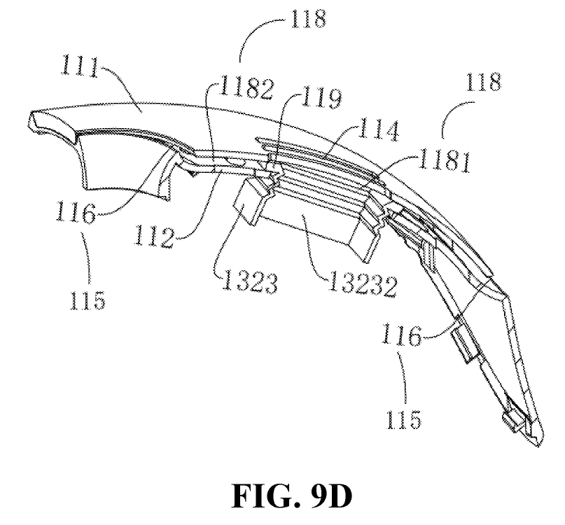

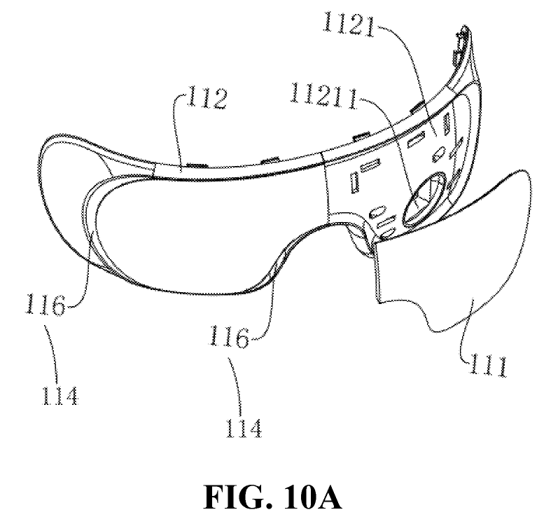

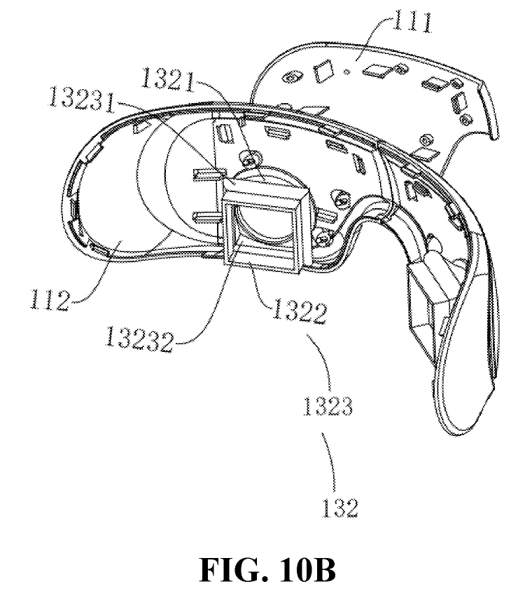

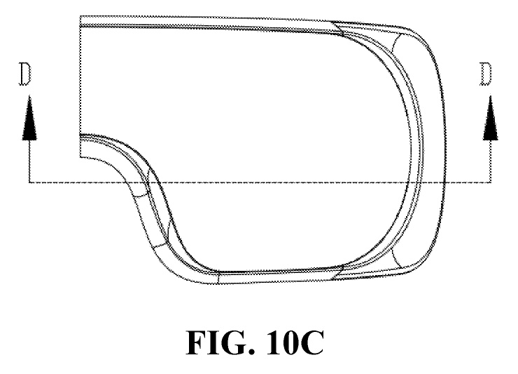

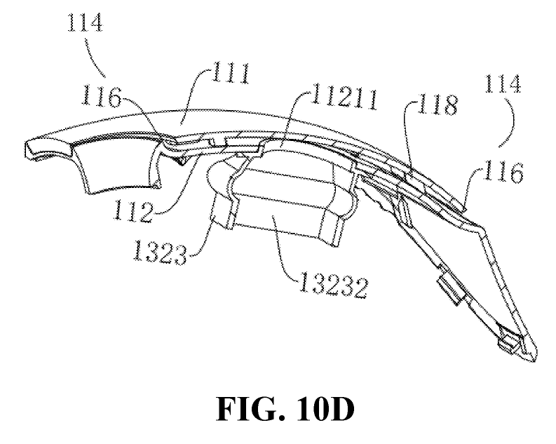

Referring to FIGS. 9B and 10B, in some embodiments, the support member 13 may be two elastic members 132. Each elastic member 132 may include a first end 1321 and a second end 1322. The first end 1321 may be connected to the body housing 11. Each elastic member 132 may be a spring, elastic sheet, spring tube, bellows, elastic rubber sleeve, etc. The second end 1322 of the elastic member 132 may be directly or indirectly connected to the respective cold and hot compress assembly 12. The elastic member 132 can extend, contract, and deflect, enabling a contact position between the cold and hot compress assembly 12 and the eye and periorbital area and an angle of the cold and hot compress assembly 12 relative to the eye and periorbital area to be dynamically adjusted.

Referring to FIGS. 2, 4, 9B, and 10B, in some embodiments, the support member 13 may include a flexible contact layer 131 and elastic members 132. The first end 1321 of each elastic member 132 may pass through the inner face 1132 and may be mounted to the body housing 11. The cold and hot compress assembly 12 may pass through the contact layer 131 and may be directly or indirectly connected to the second end 1322 of a respective elastic member 132. When pressure from the eyes and periorbital areas acts on the cold and hot compress assemblies 12, the pressure may be first transmitted to the contact layer 131. The contact layer 131 may use an elasticity of a material of the contact layer 131 for initial cushioning and perform a preliminary compression adaptation according to the contours of the eyes and periorbital areas, such as compressing more at protruding parts of the eyes and periorbital areas and compressing less at recessed parts of the eyes and periorbital areas. Subsequently, the pressure may be further transmitted to the elastic members 132. The elastic members 132 may extend, contract, and deflect, while the contact layer 131 may deform locally and laterally to cooperate with movement of the elastic members 132, cooperatively guiding movements of the cold and hot compress assemblies 12 to ensure the cold and hot compress assemblies 12 can fit the eyes and periorbital areas. After the user removes the eye massager, the pressure may disappear, and the elastic members 132 may return to initial positions and states relying on elastic restoring forces of the elastic members 132, and the contact layer 131 may gradually recover an original shape.

In some embodiments, the first end 1321 of each elastic member 132 may be mounted to the inner face 1132. In this case, the elastic members 132 may be completely exposed from the body housing 11 and can deform freely. The contact layer 131 may sleeve an outer side of the elastic members 132. When the elastic members 132 deform, the contact layer 131 may deform correspondingly.

Referring to FIGS. 9B and 10B, in other embodiments, the first end 1321 of each elastic member 132 may extend into the body housing 11 and may be fixed to an internal structure of the body housing 11. In this case, an activity space may be defined at least between the second end 1322 of the elastic member 132 and the body housing 11. The activity space may provide necessary physical clearance for the extension, contraction, and deflection of the elastic member 132, avoiding an interference with the body housing 11. The activity space may also effectively prevent the elastic member 132 from friction, collision, or squeezing with the body housing 11 during repeated extension, contraction, and deflection.

Referring to FIGS. 9B and 10B, in some embodiments, each elastic member 132 may be a telescopic sleeve 1323 which may be made of an elastic material. The telescopic sleeve 1323 may include a side wall 13231. The side wall 13231 may at least partially have pleats or a curved face. The pleats or curved face can extend and contract. The pleats or curved face on the side wall 13231 may act as preset deformation areas. The telescopic sleeve 1323 is capable of elastic deformation in response to an external force. The pleats or curved face of the telescopic sleeve 1323 may unfold or fold, thereby allowing the telescopic sleeve 1323 to extend and contract; the pleats and curved faces may also bend, causing the second end 1322 of the elastic member 132 to deflect. When the pressure decreases, the pleats or curved face may recover an original shape under an action of the elastic material. An unfolding and recovery of the pleats or curved face may be a gradual and gentle process, which can effectively absorb and buffer pressure fluctuations, improving user comfort when wearing the eye massager. The telescopic sleeve 1323 may be made of elastic materials such as silicone, rubber, etc.

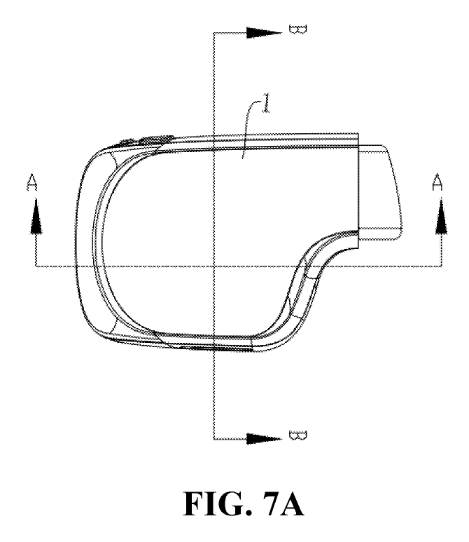

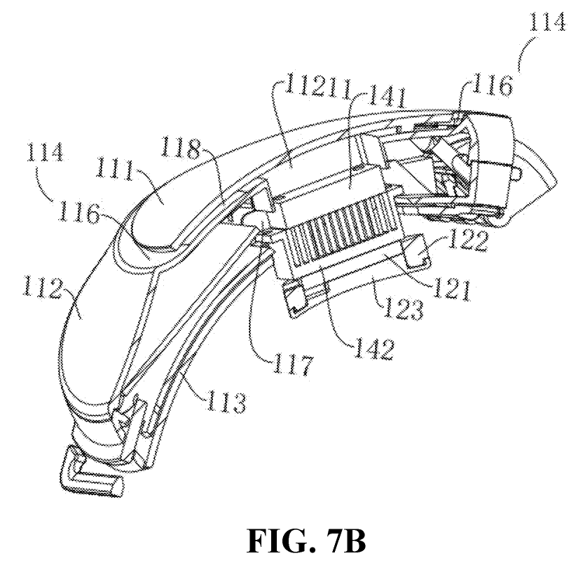

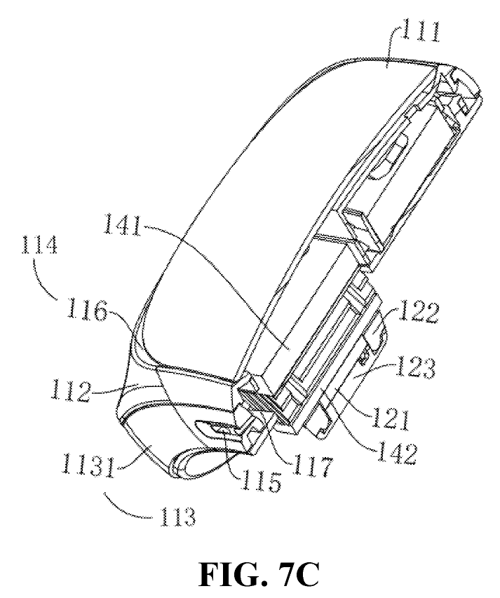

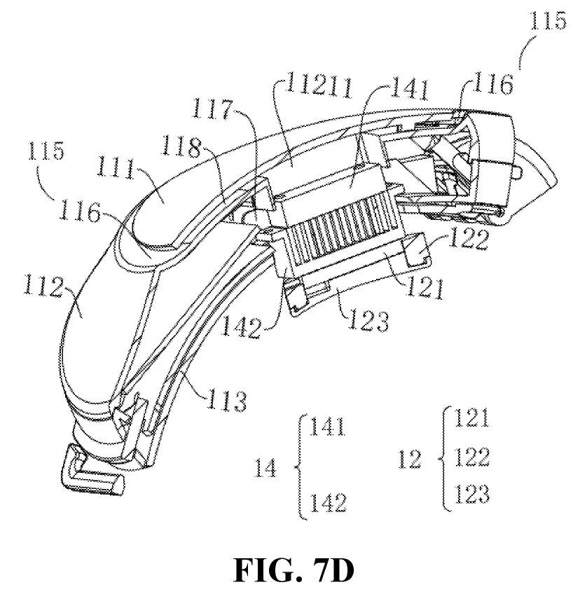

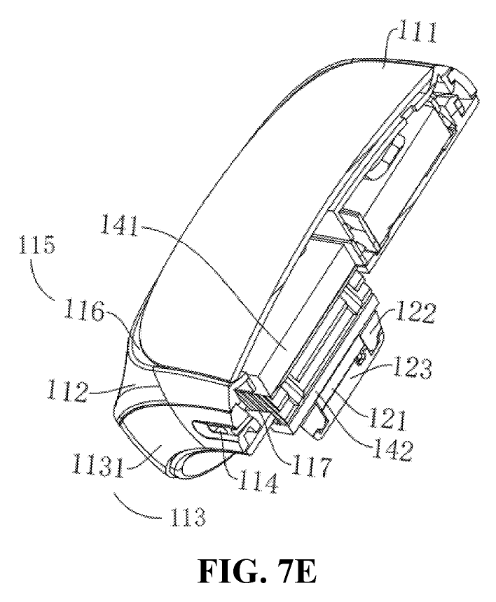

Referring to FIGS. 9B, 10B, and 7B, in some embodiments, the elastic member 132 may be a telescopic sleeve 1323 which may be made of an elastic material. A first air passage 13232 may be formed in the telescopic sleeve 1323. Both the first end 1321 and the second end 1322 of the telescopic sleeve 1323 may be open, to communicate with the first air passage 13232. The first end 1321 of the telescopic sleeve 1323 may be communicated with an outside of the eye massager. The eye massager further may include a heat dissipation assembly 14. The heat dissipation assembly 14 may be arranged adjacent to the second end 1322 of the telescopic sleeve 1323. The cold and hot compress assembly 12 may be arranged adjacent to the heat dissipation assembly 14. When the heat dissipation assembly 14 operates, external air flow may enter from the first end 1321 of the telescopic sleeve 1323 into the first air passage 13232, then reach the second end 1322 through the first air passage 13232, pass through the heat dissipation assembly 14, and carry away excess heat generated by the cold and hot compress assembly 12. In the present disclosure, the elastic member 132 may be arranged as an elastic telescopic sleeve 1323 which may combine elastic support and an air passage into one, simplifying a structure of the eye massager, and ensuring a continuous heat dissipation from the heat dissipation assembly 14 to the cold and hot compress assembly 12, regardless of how the cold and hot compress assembly 12 moves.

The heat dissipation assembly 14 may include a fan 141 and a heat-dissipating member 142. The fan 141 may be arranged close to or attached to the heat-dissipating member 142. The fan 141 may be an axial flow fan, centrifugal fan, mixed flow fan, etc. The fan 141 and the heat-dissipating member 142 may be arranged opposite each other, as long as the heat-dissipating member 142 is close to an air outlet of the fan 141 and may be cooled by the fan 141. The heat-dissipating member 142 may be arranged close to or attached to the thermoelectric member 121. The heat-dissipating member 142 may exchange heat with the thermoelectric member 121. When the fan 141 operates, the fan 141 can draw external air flow into the body housing 11, and the external air flow may pass through the heat-dissipating member 142 and then discharged. The external air flow may carry away heat from the heat-dissipating member 142, ultimately achieving heat dissipation for the thermoelectric member 121.

In other embodiments, the fan 141 may not be provided. Heat dissipation may be achieved through natural air circulation via the heat-dissipating member 142 and air passages. Some internal components may be made of materials with good thermal conductivity to improve heat dissipation efficiency.

Referring to FIGS. 9B, 10B, and 7B, when the first air passage 13232 is formed inside the telescopic sleeve 1323, the fan 141 may be an axial flow fan. The fan 141 and the heat-dissipating member 142 may be stacked and mounted to the second end 1322 of the telescopic sleeve 1323. The thermoelectric member 121 may be arranged close to the heat-dissipating member 142. The fan 141, heat-dissipating member 142, and cold and hot compress assembly 12 may be stacked sequentially along a length direction of the telescopic sleeve 1323, thus ensuring a shortest heat transfer path and a highest heat transfer efficiency.

It can be understood that the fan 141 and the heat-dissipating member 142 may also be arranged separately and independently. In this case, the fan 141 may be a centrifugal fan, mixed flow fan, etc. The fan 141 may be located outside the first end 1321 of the telescopic sleeve 1323, as long as the air outlet side of the fan 141 may be arranged facing the first end 1321 of the telescopic sleeve 1323, enabling external air flow to enter the first air passage 13232.

In other embodiments, the elastic member 132 may also be a spring, elastic sheet. Besides, the support member 13 may also be only a flexible contact layer 131. In this case, the fan 141 may be an axial flow fan 141, centrifugal fan 141, mixed flow fan 141, etc. The fan 141 may cooperate with air passages inside the body housing 11, as long as the external air flow can carry away heat from the cold and hot compress assembly 12.

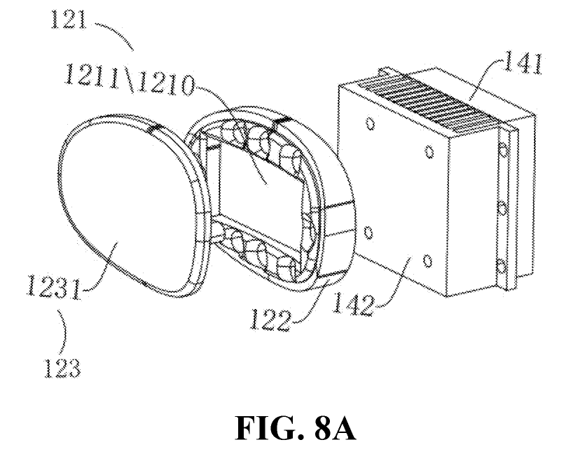

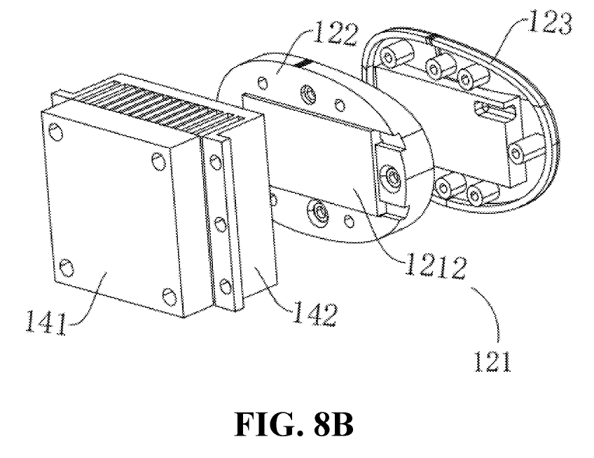

Referring to FIGS. 8A and 8B, in some embodiments, the cold and hot compress assembly 12 may further include a base 122. The thermoelectric member 121 may be formed as a sheet. The thermoelectric member 121 may include a first face 1211 and a second face 1212 which may be arranged opposite each other along a thickness direction of the thermoelectric member 121. The base 122 may be arranged to surround the thermoelectric member 121. The first face 1211 and the second face 1212 may be exposed from the base 122. The heat conduction member 123 may be mounted to the base 122. At least a part of the heat conduction member 123 can exchange heat with the first face 1211 of the thermoelectric member 121. The first face 1211 may form the first working portion 1210. The heat conduction member 123 can conduct the cold or heat generated by the thermoelectric member 121 to the respective eye and periorbital area. The heat conduction member 123 may directly contact the first face 1211 of the thermoelectric member 121. In an example, the cold and hot compress assembly 12 may be completely embedded in a filler of the contact layer 131, the heat conduction member 123 may be wrapped by an outer fabric and contact the human body through the outer fabric to achieve cold or hot compress. In another example, the cold and hot compress assembly 12 may be partially embedded in the filler of the contact layer 131, and the heat conduction member 123 may pass through openings in the outer fabric to directly contact the human body. The heat conduction member 123 may also indirectly contact the first face 1211 of the thermoelectric member 121. The base 122 and the thermoelectric member 121 may be embedded in the filler of the contact layer 131. Each of the heat conduction member 123 and the base 122 may be arranged on a respective one of two sides of the outer fabric. The outer fabric may be pressed by an operation of the heat conduction member 123 and the base 122. The heat conduction member 123 and the base 122 may be secured to each other by fasteners such as screws. The heat conduction member 123 may contact the first face 1211 of the thermoelectric member 121 through the outer fabric.

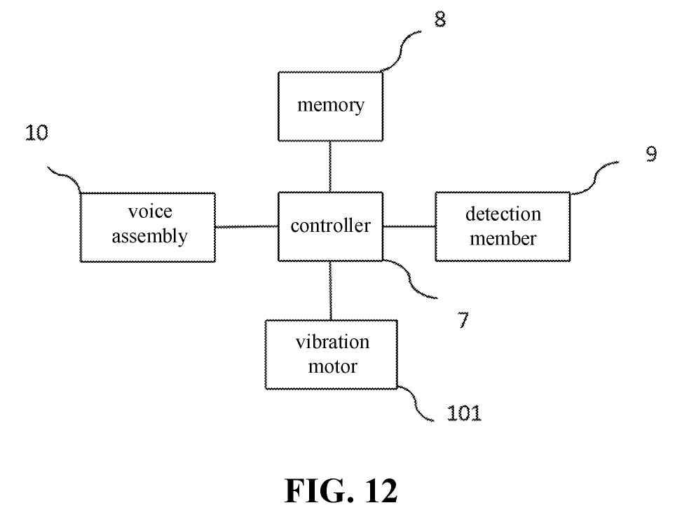

Further referring to FIG. 12, the body assembly 1 may further include a controller 7 and a memory 8. The controller 7 may be electrically connected to the thermoelectric member 121 and the memory 8. The memory 8 may store a plurality of preset temperatures. The preset temperatures may be set according to different usage modes and needs, such as cold compress mode, hot compress mode, soothing mode, etc. The user may input a user instruction via voice or buttons, such as selecting a soothing mode, or selecting a level for cold compress or hot compress. The controller 7 may call a corresponding preset temperature from the memory 8 and control the thermoelectric member 121 to start a cooling or heating function until reaching the preset temperature. The arrangement of the controller 7 and the memory 8 may enable the eye massager to provide a precise and flexible temperature adjustment function. Additionally, the preset temperatures may also be a maximum range values for cooling and heating temperatures. For example, during heating, the preset temperature may be 36° C. -40° C. ; during cooling, the preset temperature may be 15° C. -19° C. , avoiding scalding the user from overheating or freezing the user from excessive cold.

Further referring to FIG. 12, the body assembly 1 further may include a detection member 9. The detection member 9 may be arranged close to the thermoelectric member 121 or the heat conduction member 123, for a real-time detection of a temperature of the thermoelectric member 121 or the heat conduction member 123. The detection member 9 may include a temperature sensor. The temperature sensor may collect current temperature data of the thermoelectric member 121 or the heat conduction member 123, convert the temperature data into a temperature signal, and transmit the temperature signal to the controller 7 for processing. The controller 7 may dynamically adjust an operating state of the thermoelectric member 121 according to the received temperature signal, thereby precisely adjusting the cooling or heating function. For example, when the detection member 9 detects that the temperature of the thermoelectric member 121 or the heat conduction member 123 may be too high, the controller 7 may reduce a heat output of the thermoelectric member 121 to prevent overheating; when the temperature may be too low, the controller 7 may increase the heat output of the thermoelectric member 121 to ensure rapid achievement of the preset temperature. Optionally, the detection member 9 may include two temperature sensors respectively attached/embedded on left and right sides of the thermoelectric member 121, for detecting the real-time temperature of the thermoelectric member 121.

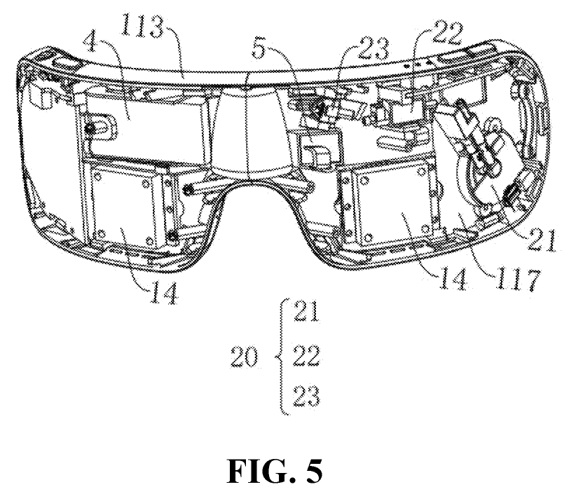

Referring to FIGS. 2-6, in some embodiments, the body assembly 1 further may include a massage assembly 2. The massage assembly 2 may include an inflation/deflation assembly 20 and an air bag 24. The inflation/deflation assembly 20 may include an air pump 21, one or more air valves 22, and one or more nozzles 23. The air pump 21, the one or more air valves 22, and the one or more nozzles 23 may be located in the body housing 11. The air pump 21 may be communicated to the one or more air valves 22 via a flexible tube. The one or more air valves 22 may be respectively communicated to one or more nozzles 23 via flexible tubes. The air bag 24 may be mounted on the inner face 1132 of the body housing 11. The air bag 24 may include one or more compartments. The one or more nozzles 23 may be respectively communicated to one or more compartments. The one or more air valves 22 enable the air pump 21 to selectively inflate specific compartments of the air bag 24 of the eye massager, or extract air from specific compartments of the air bag 24. The repeated inflation and deflation process may provide a massaging effect on the human body.

The body housing 11 further may include an outer face 1134 opposite to the inner face 1132. The cold and hot compress assemblies 12 may be spaced apart from the air bag 24 in a direction extending from the outer face 1134 toward the inner face 1132, causing the cold and hot compress assemblies 12 to avoid the air bag 24, to prevent the heat generated by the cold and hot compress assemblies 12 from affecting a service life of the air bag 24. Additionally, in the direction extending from the outer face 1134 toward the inner face 1132, projections of the cold and hot compress assemblies 12 and the air bag 24 on the inner face 1132 may not be overlapped with each other, which may also cause the cold and hot compress assemblies 12 to avoid the air bag 24. In some embodiments, the air bag 24 may be located above the cold and hot compress assemblies 12 and arranged horizontally. Both ends of the air bag 24 may be bent downward relative a middle part of the air bag 24 and extend to both sides of the eye massager. After the user wears the eye massager, the air bag 24 may cover areas such as the user’s upper eyelids, forehead, and positions at both temples. Through inflation and deflation adjustments, the air bag 24 can provide targeted air pressure massage, soothing the temples, areas around the eyebrows, and eye areas.

Referring to FIG. 8A, in some embodiments, the heat conduction member 123 may include a working face 1231 for exchanging heat with the human body. The working face 1231 may be curved, a curvature of the working face 1231 may match the respective eye and periorbital area, to increase a fitting degree between the working face 1231 and the respective eye and periorbital area.

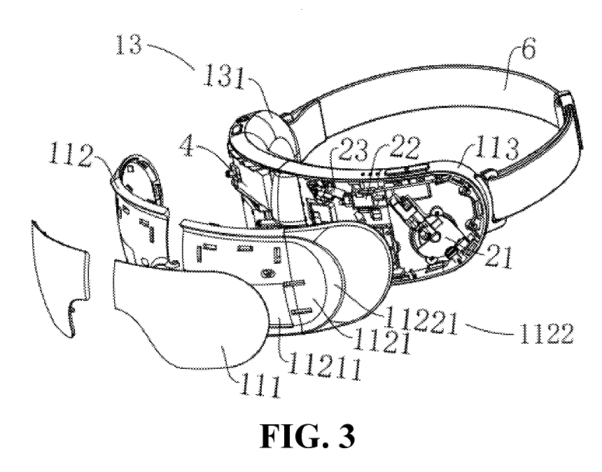

Referring to FIGS. 3, 4, 7A-7E, and 9A-11C, in some embodiments, at least one first air vent 114 and at least one second air vent 115 may be formed on the body housing 11. The body housing 11 may include a first housing 111, a second housing 112, and a third housing 113. The second housing 112 and the third housing 113 may be assembled to cooperatively define an accommodation cavity 117. The inner face 1132 of the body housing 11 may be a side of the third housing 113 used to face the human body. The first housing 111, as an appearance part, may be mounted to a side of the second housing 112 used to face away from the human body. At least one of the first air vent 114 and the second air vent 115 may be formed by a first gap 116 formed between a partial edge of the first housing 111 and the second housing 112, to achieve a hole-free appearance and enhance the aesthetic feel of the product. The heat dissipation assembly 14 may be arranged close to or attached to the cold and hot compress assembly 12, transferring the excess heat generated by the cold and hot compress assembly 12 to the heat dissipation assembly 14. When the heat dissipation assembly 14 operates, the external air flow may enter from the first air vent 114, pass through the heat dissipation assembly 14, and then may be discharged from the second air vent 115. The external air flow can carry away heat from the heat dissipation assembly 14, completing effective heat exchange.

Referring to FIGS. 3 and 7B, in some embodiments, the second housing 112 may include a bearing face 1121 arranged facing the first housing 111 and a flow guide member 1122 protruding from and surrounding an outer periphery of the bearing face 1121. The first housing 111 may be mounted to the bearing face 1121, and a second gap 118 may be formed between the first housing 111 and the bearing face 1121. One of the first housing 111 and the bearing face 1121 may be arranged with a plurality of hooks, and the other of the first housing 111 and the bearing face 1121 may define a plurality of mounting holes. The plurality of hooks may be mounted in the mounting holes one by one to mount the first housing 111 to the bearing face 1121. The flow guide member 1122 may protrude toward the first housing 111, and at least a part of the flow guide member 1122 may extend obliquely outward relative to the bearing face 1121 to form a flow guide face 11221. The flow guide face 11221 and a portion of an edge of the first housing 111 may cooperatively form the first gap 116. Other parts of the flow guide member 1122 may be sealed to the first housing 111. When the heat dissipation assembly 14 operates, the external air flow may be guided by the flow guide face 11221, naturally changing direction to pass through the first gap 116. The arrangement of the flow guide face 11221 can reduce wind resistance and noise generated when the external air flow enters the first gap 116.

Referring to FIGS. 1 and 3, in some embodiments, the first housing 111 may include an outer face 1111 used to face away from the human body. An upper edge of the flow guide face 11221 may be located on an extension plane of the outer face 1111 or close to the extension plane of the outer face 1111, thereby ensuring a smooth and uninterrupted transition from the outer face 1111 of the first housing 111 to the flow guide face 11221. When viewed from a front and side of the eye massager, the first gap 116 may be hidden, giving the eye massager a sense of integration and simplicity.

Referring to FIGS. 1, 3, and 7B, in some embodiments, the body housing 11 may include an outer edge used to fit the human temples and an inner edge used to fit the human nose. The first gap 116 may be formed at an outer edge of the first housing 111, and/or the first gap 116 may be formed at an inner edge of the first housing 111. Since the eye massager may be used for eye massage, and two body assemblies 1 may be arranged, for the entire eye massager, two first gaps 116 may be formed, the two first gaps 116 may be respectively formed at left and right outer edges of the first housing 111, and the two first gaps 116 may also be respectively formed at the left and right inner edges of the first housing 111. Four first gaps 116 may also be formed, and the four first gaps 116 may be respectively formed at the left and right outer edges and the left and right inner edges of the first housing 111. When the user wears the eye massager, two of the first gaps 116 may be respectively close to the user’s cheekbones, and the other two first gaps 116 may be respectively close to both sides of the user’s nasal bridge. In this case, viewed from the side of the eye massager, the first gaps 116 form a natural transition with the user’s face without abruptness; viewed from the front of the eye massager, the first gaps 116 may be blocked by the user’s nose.

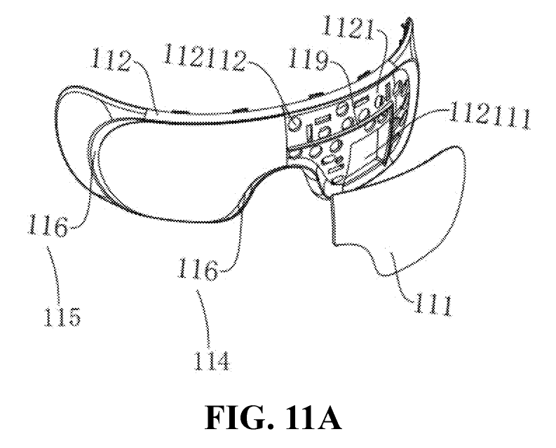

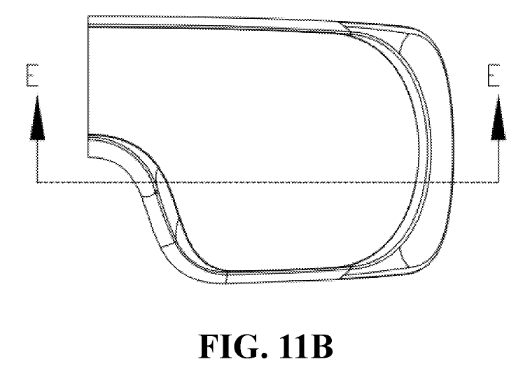

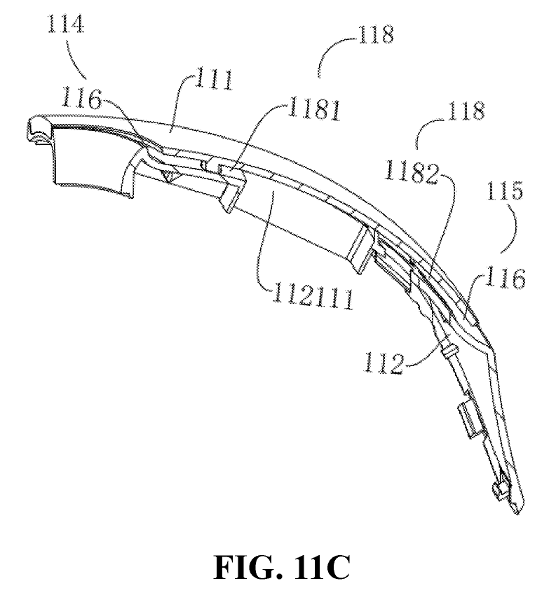

Referring to FIGS. 7A-7E and 8A-11C, in some embodiments, the second housing 112 may include a bearing face 1121. The first housing 111 may be mounted on the bearing face 1121. A second gap 118 may be formed between the first housing 111 and the bearing face 1121. At least one opening 11211 may be formed on the bearing face 1121, for communicating the second gap 118 with the accommodation cavity 117. The first air vent 114 and the second air vent 115 may each be formed as at least one. The first air vent 114 may be communicated with one of the second gap 118 and the accommodation cavity 117, and the second air vent 115 may be communicated with the other one of the second gap 118 and the accommodation cavity 117; or the first air vent 114 may be communicated with the second gap 118, and the second air vent 115 may be communicated with both the second gap 118 and the accommodation cavity 117. That is, there may be three cases. The first case, as shown in FIGS. 7B-7C and 10A-10D, the first air vent 114 may be communicated with the second gap 118 for air intake, and the second air vent 115 may be communicated with the accommodation cavity 117 for air exhaust. The second case, as shown in FIGS. 7D-7E, the first air vent 114 may be communicated with the accommodation cavity 117 for air intake, and the second air vent 115 may be communicated with the second gap 118 for air exhaust. The third case, as shown in FIGS. 9A-9D and 11A-11C, the first air vent 114 may be communicated with the second gap 118 for air intake, and the second air vent 115 may be communicated with both the second gap 118 and the accommodation cavity 117 for air exhaust.

Specifically, as shown in FIGS. 7B-7C and 10A-10D, in one embodiment, the first air vent 114 may be formed by the first gap 116. For a single body assembly 1, one or two first air vents 114 may be formed. The first air vent 114 may be communicated with the second gap 118 for air intake. The third housing 113 may include a peripheral side wall 1131. The second air vent 115 may be formed on the peripheral side wall 1131. The second air vent 115 may be communicated with the accommodation cavity 117. The fan 141 of the heat dissipation assembly 14 may be located in the accommodation cavity 117. When the fan 141 operates, external air flow may enter the second gap 118 through the first air vent 114, enter the accommodation cavity 117 through the opening 11211 on the bearing face 1121, carry away heat from the heat-dissipating member 142, and then may be discharged through the second air vent 115.

As shown in FIGS. 7D-7E, in another embodiment, positions of the first air vent 114 and the second air vent 115 may be swapped. The first air vent 114 may be formed on the peripheral side wall 1131 of the third housing 113 and may be communicated with the accommodation cavity 117. The second air vent 115 may be formed by the first gap 116. The second air vent 115 may be communicated with the second gap 118. When the fan 141 operates, external air flow may enter the accommodation cavity 117 through the first air vent 114, carry away heat from the heat-dissipating member 142, then enter the second gap 118 through the opening 11211 on the bearing face 1121, and may be discharged through the second air vent 115.

As shown in FIGS. 9A-9D and 11A-11C, in some embodiments, the body housing 11 further may include a shielding portion 119. The shielding portion 119 may extend from the bearing face 1121 toward the first housing 111 and may be connected with the first housing 111. The shielding portion 119 may form a partition or duct and partition the second gap 118 to form a second air passage 1181 and third air passage 1182 which may be mutually independent from each other. The at least one opening 11211 may include a first opening 112111 and a second opening 112112 which may be formed on the bearing face 1121. Two first gaps 116 may be formed. The first air vent 114 may be formed by one of the two first gaps 116. The first air vent 114, the second air passage 1181, and the first opening 112111 may be communicated with each other sequentially for air intake. The at least one second air vent 115 may be formed by the other first gap 116. The second opening 112112, the third air passage 1182, and the at least one second air vent 115 may be communicated with each other sequentially for air exhaust. The arrangement of the shielding portion 119 can partition the second gap 118, allowing both the first air vent 114 and the second air vent 115 to be formed by the first gap 116. Additionally, the second air vent 115 may also be formed on the peripheral side wall 1131 of the third housing 113, cooperating with the second air vent 115 formed by the first gap 116 for air exhaust.

As shown in FIGS. 9A-9D, in one embodiment, the first air vent 114 may be formed on the first housing 111. The shielding portion 119 may be hollow inside and both ends may be open, to enclose and form the second air passage 1181 inside. Both ends of the shielding portion 119 may be directly communicated to the first air vent 114 and the first opening 112111, causing the second air passage 1181 to be communicated with the first air vent 114 and the first opening 112111. One or more first gaps 116 may be formed. The at least one second air vent 115 may be formed by the first gap 116. When the fan 141 operates, external air flow may enter the accommodation cavity 117 through the first air vent 114 and the second air passage 1181 inside the shielding portion 119, carry away heat from the heat-dissipating member 142, then the air flow may enter the third air passage 1182 through the second opening 112112 on the bearing face 1121, and may be discharged through the second air vent 115 formed by the first gap 116. Another second air vent 115 may also be formed on the peripheral side wall 1131 of the third housing 113, and the air flow may also be discharged through the second air vent 115 on the peripheral side wall 1131.

As shown in FIGS. 11A-11C, in another embodiment, the shielding portion 119 may be arranged as an L-shaped partition as shown in the figures, partitioning the second gap 118 to form two independent regions. The two independent regions form the second air passage 1181 and the third air passage 1182 respectively. Two first gaps 116 may be formed. The first air vent 114 may be formed by one of the two first gaps 116. At least one second air vent 115 may be formed by the other of the two first gap 116. When the fan 141 operates, external air flow may enter the accommodation cavity 117 through the first air vent 114 and the second air passage 1181, carry away heat from the heat-dissipating member 142, then the air flow may enter the third air passage 1182 through the second opening 112112 on the bearing face 1121, and may be discharged through the second air vent 115 formed by the first gap 116. Another second air vent 115 may also be formed on the peripheral side wall 1131 of the third housing 113, and the air flow may also be discharged through the second air vent 115 on the peripheral side wall 1131.

Referring to FIG. 9B, in some embodiments, as mentioned above, the support member 13 may be a telescopic sleeve 1323 which may be elastic. A first air passage 13232 may be formed in the telescopic sleeve 1323. Both a first end 1321 and a second end 1322 of the telescopic sleeve 1323 may be open, to be communicated with the first air passage 13232. Optionally, the telescopic sleeve 1323 may be integrally formed with the shielding portion 119 as a one-piece structure. The shielding portion 119 may extend from the second end 1322 of the telescopic sleeve 1323 and extend into the first opening 112111 on the bearing face 1121. An end of the shielding portion 119 away from the telescopic sleeve 1323 may be communicated with the first air vent 114 formed on the first housing 111. The heat dissipation assembly 14 may be mounted to the second end 1322 of the telescopic sleeve 1323. When the fan 141 operates, external air flow may directly enter the shielding portion 119 and the telescopic sleeve 1323 through the first air vent 114, and may be directionally conveyed to a heat exchange area where the heat dissipation assembly 14 is located through the shielding portion 119 and the telescopic sleeve 1323.

In some embodiments, the air pump 21 may be a flat air pump. Compared to traditional block-type air pumps 21, the flat air pump 21 can greatly save a mounting space. Moreover, through a cooperation of the contact layer 131 and the support members 13, an adaptive adjustment of the heat conduction member 123 to the user’s corresponding area for cold/hot compress may be achieved, without needing to add additional elastic structures (such as springs or other elastic structures), effectively and reasonably utilizing a mutual cooperation of internal structures and simplifying an overall mounting structure. In addition, by arranging the first gap 116, the second gap 118, the shielding portion 119, and the opening 11211 on the body housing 11, an effective ventilation and heat dissipation structure may be formed without adding separately arranged air passage structures. The comprehensive design and reasonable arrangement of the above structures enable a volume of the entire eye massager to be effectively and maximally reduced, the body assembly 1 to be thinner, and an overall weight to be lighter. When the user wears and uses the eye massager, a sense of gravity and foreign body assembly 1 may be lighter, greatly improving user experience.

In some embodiments, the eye massager may further may include one or more vibration motors 101. The one or more vibration motors 101 may be mounted in the accommodation cavity 117. When a plurality of vibration motors 101 are provided, the plurality of vibration motors 101 may be arranged in a specific order and may operate according to a specific time sequence to form vibration trajectories, used to simulate massage techniques such as kneading and rolling.

In some embodiments, the eye massager may further include a battery 4 and a voice assembly 10. The battery 4 can supply power to the controller 7, the memory 8, the voice assembly 10, the cold and hot compress assemblies 12, the heat dissipation assemblies 14, the massage assembly 2, and the one or more vibration motors 101. The controller 7 may be electrically connected to each of the voice assembly 10 and the memory 8. The voice assembly 10 may include a voice recognition circuit, a microphone, and a speaker. The cold and hot compress assemblies 12, the heat dissipation assemblies 14, the massage assembly 2, and the vibration motors 101 have a plurality of working modes. The memory 8 may store preset instructions corresponding to the working modes. The user may input a user instruction via voice. The microphone may transmit the user instruction to the voice recognition circuit. The voice recognition circuit may convert the received user instruction into an electrical signal and transmit the electrical signal to the controller 7. The controller 7 may compare the user instruction with the preset instructions in the memory 8. When the user instruction matches one of the preset instructions, the controller 7 may control the heat dissipation assemblies 14, the massage assembly 2, or the vibration motors 101 to operate in a corresponding working mode. The speaker 5 may generate voice replying to user instruction and may play music, etc. In addition to voice control, a plurality of buttons 1133 may be arranged on a top portion of the body housing 11. The plurality of buttons 1133 may include a power button, cold/hot compress touch buttons, massage mode control buttons, etc. Particularly, shapes and sizes of the buttons 1133 may be significantly different from each other, and the buttons 1133 may be distributed on the left and right sides of the eye massager. After the user puts on the eye massager, the buttons 1133 may be close to the user’s outer eye corners, making it easier for the user to touch the buttons 1133 and distinguish between buttons of different functions.

In some embodiments, the eye massager may further include a strap 6. Each of two ends of the body housing 11, which may be arranged away from each other, may be arranged with a fastening member 1110. The fastening member 1110 may include a buckle, snap fastener, slide fastener, etc. Both ends of the strap 6 may be respectively connected to the two fastening members 1110. The user can wear the eye massager via the strap 6. The fastening member 1110 may be arranged in a hook shape. An end of the strap 6 may slide in a gap formed in the fastening member 1110. The hook-shaped fastening member 1110 can ensure the strap 6 may not slip out when the air bag 24 is inflated.

In the present disclosure, the eye massager may further have a memory function. When the eye massager is turned off, the controller 7 and the memory 8 may save user-defined configurations such as an air pressure level of the massage assembly, a temperature selection of the cold and hot compress assembly, timing settings, and built-in music settings. The next time the eye massager is turned on, the system may automatically remember relevant settings from the last power-on, allowing the user to continue using the previous settings without readjusting. Through the memory function, users can enjoy a continuous usage experience more easily without frequently adjusting parameters.

In some embodiments, the eye massager may further have a protection mechanism. When the user simultaneously chooses to turn off both the cold and hot compress assemblies 12 and the massage assembly 2, and does not perform any operation within a preset time, for example, within 30 seconds, the eye massager may automatically enter a protective shutdown mode to prevent unnecessary energy consumption or potential faults from the device being in an inoperative state for a long time. Furthermore, when the user shuts down manually or via automatic timing while the cold and hot compress assemblies 12 are operating, the eye massager may take additional heat dissipation protection measures. In this case, fans 141 of the heat dissipation assemblies 14 may continue to operate for a preset duration, for example, 30 seconds, continuing to dissipate heat for the cold and hot compress assemblies 12 to avoid scalding the user due to sudden shutdown and overheating of the cold and hot compress assemblies 12.

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

What is claimed is:

1. An eye massager, comprising at least one body assembly, the at least one body assembly comprising:
a body housing, defining at least one first air vent and at least one second air vent and comprising an inner face, a first housing, and a second housing, wherein the first housing is mounted to an outer side of the second housing, and at least one of the first air vent and the second air vent is defined by a first gap formed between the first housing and the second housing; the at least one first air vent functions as an air inlet, and the at least one second air vent functions as an air outlet;
a cold and hot compress assembly, comprising:
a thermoelectric member, comprising a first working portion, wherein changing a current direction in the thermoelectric member enables the first working portion to cool or heat; and
a heat conduction member, configured to at least partially exchange heat with the first working portion, and apply cold or hot compress to the user; and
a heat dissipation assembly, at least partially arranged in the body housing, and configured to dissipate excess heat from the cold and hot compress assembly.

2. The eye massager according to claim 1, wherein the body assembly further comprises a support member, the support member is mounted to the body housing, the cold and hot compress assembly is directly or indirectly mounted to the support member, and the support member is deformable to drive the cold and hot compress assembly to move.

3. The eye massager according to claim 2, wherein the support member comprises a contact layer which is flexible, the contact layer is attached to the inner face of the body housing, and the cold and hot compress assembly is at least partially embedded in the contact layer.

4. The eye massager according to claim 2, wherein the support member comprises an elastic member, the elastic member comprises a first end and a second end, the first end is connected to the body housing, and the second end is directly or indirectly connected to the cold and hot compress assembly.

5. The eye massager according to claim 4, wherein the elastic member is configured as a telescopic sleeve which is elastic, the telescopic sleeve comprises a side wall, the side wall is at least partially arranged with pleats or a curved face, and the pleats or the curved face are capable of telescoping and bending.

6. The eye massager according to claim 4, wherein the elastic member is configured as a telescopic sleeve which is elastic, the telescopic sleeve defines a first air passage, each of the first end and the second end is open and is communicated with the first air passage, the first end is communicated with an outside of the eye massager, the heat dissipation assembly is arranged adjacent to the second end, and the cold and hot compress assembly is arranged adjacent to the heat dissipation assembly.

7. The eye massager according to claim 1, wherein the heat dissipation assembly comprises a fan and a heat-dissipating member, the fan is arranged close to or attached to the heat-dissipating member, and the heat-dissipating member is arranged close to or attached to the thermoelectric member to exchange heat with the thermoelectric member.

8. The eye massager according to claim 1, wherein the cold and hot compress assembly further comprises a base, the thermoelectric member comprises a first face and a second face, the base is arranged to surround an outer periphery of the thermoelectric member, and each of the first face and the second face is exposed from the base;
at least a part of the heat conduction member is configured to exchange heat with the first face, and the first face is configured to form the first working portion.

9. The eye massager according to claim 1, wherein the heat conduction member comprises a second working portion configured to exchange heat with the user, and the second working portion has a curvature matching an eye and periorbital area.

10. The eye massager according to claim 1, wherein the body assembly further comprises a controller and a memory, the controller is electrically connected to the thermoelectric member and the memory, the memory stores a plurality of preset temperatures, and the controller is configured to call a corresponding one of the plurality of preset temperatures according to a user instruction and control the thermoelectric member to cool or heat.

11. The eye massager according to claim 10, wherein the body assembly further comprises a detection member, the detection member is arranged close to the thermoelectric member or the heat conduction member, the detection member is configured to detect a temperature of the thermoelectric member or the heat conduction member, generate a temperature signal, and transmit the temperature signal to the controller, and the controller is configured to adjust an operating state of the thermoelectric member based on the temperature signal.

12. The eye massager according to claim 1, wherein the body assembly further comprises a massage assembly, the massage assembly comprises an inflation/deflation assembly and an air bag; the inflation/deflation assembly is arranged in the body housing, the body housing comprises an outer face arranged opposite to the inner face, the air bag is arranged on the inner face, and the inflation/deflation assembly is communicated with the air bag and is configured to inflate or deflate the air bag; the cold and hot compress assembly is spaced apart from the air bag in a direction extending from the outer face toward the inner face.

13. The eye massager according to claim 1, wherein the body housing further comprises a third housing, the second housing and the third housing cooperatively define an accommodation cavity;
when the heat dissipation assembly operates, external air flow enters from the first air vent, passes through the heat dissipation assembly, and then is discharged from the second air vent.

14. The eye massager according to claim 1, wherein the second housing comprises a bearing face and a flow guide member protruding from the bearing face and surrounding an outer periphery of the bearing face, the first housing is mounted to the bearing face, at least a part of the flow guide member extends obliquely outward relative to the bearing face to form a flow guide face, the flow guide face and an edge of the first housing cooperatively define the first gap, and other parts of the flow guide member is sealed to the first housing.

15. The eye massager according to claim 14, wherein the first housing comprises an outer face facing away from the inner face, and an upper edge of the flow guide face is arranged on an extension plane of the outer face or close to the extension plane of the outer face.

16. The eye massager according to claim 1, wherein the first gap is arranged at an outer edge of the first housing, and/or the first gap is located at an inner edge of the first housing.

17. The eye massager according to claim 1, wherein the second housing comprises a bearing face, the first housing is mounted to the bearing face, and a second gap is formed between the first housing and the bearing face; at least one opening is formed on the bearing face, and the second gap and the accommodation cavity are communicated with each other via the at least one opening.

18. The eye massager according to claim 17, wherein the first air vent is formed by the first gap, and the first air vent is communicated with the second gap; the third housing comprises a peripheral side wall, the second air vent is formed on the peripheral side wall, and the second air vent is communicated with the accommodation cavity.

19. The eye massager according to claim 17, wherein the second air vent is formed by the first gap, and the second air vent is communicated with the second gap; the third housing comprises a peripheral side wall, the first air vent is formed on the peripheral side wall or the first air vent is formed on the first housing, and the first air vent is communicated with the accommodation cavity.

20. The eye massager according to claim 17, wherein the body housing further comprises a shielding portion, the shielding portion extends from the bearing face toward the first housing and is connected with the first housing, and the shielding portion partitions the second gap to form a second air passage and a third air passage which are independent from each other;
the at least one opening comprises a first opening and a second opening, one of the first air vent and the second air vent is communicated with the second air passage and the first opening, and the other one of the first air vent and the second air vent is communicated with the second opening and the third air passage.
